(19) United States
(12) Patent Application Publication
(10) Pub. No.: US 2026/0283529 A1
(43) Pub. Date: Sep. 24, 2026

(54) METHOD AND PORTABLE DEVICE FOR MONITORING AND IDENTIFYING CARDIAC EVENTS

(71) Applicant: SMART HEALTH SOLUTIONS S.A.P.I. DE C.V., Veracruz (MX)

(72) Inventors: Miguel Eduardo ROMERO RUIZ, Veracruz (MX); Hugo Domingo GARCÍA MANILLA, Veracruz (MX); Daniel Alejandro GORDILLO HERNÁNDEZ, Veracruz (MX); Rodolfo FERRO PÉREZ, Veracruz (MX); Manuel Iván LEÓN MADRID, Veracruz (MX)

(73) Assignee: SMART HEALTH SOLUTIONS S.A.P.I. DE C.V., Veracruz (MX)

(21) Appl. No.: 18/720,138

(22) PCT Filed: Dec. 15, 2022
(86) PCT No.: PCT/IB2022/062331

§ 371 Date: Jun. 11, 2026

(30) Foreign Application Priority Data
Dec. 17, 2021 (MX) MX/A/2021/016015

(51) Int. Cl. A61B 5/332; A61B 5/00; A61B 5/308; A61B 5/346; G16H 50/20

(52) U.S. Cl. CPC A61B 5/332; A61B 5/0006; A61B 5/308; A61B 5/346; A61B 5/7267; A61B 5/746; G16H 50/20; A61B 2560/0223

(57) ABSTRACT

A biopotential signal collection and processing system for cardiac monitoring, prediction and alerts which consists of a biopotential data acquisition device fitted with a wireless module electronically connected to a regulator, which adapts the voltage of a battery. The biopotential data acquisition device also includes at least one biopotential sensor, which collects cardiac biopotential signals (ECG) that are delivered to the wireless module, adjusts, codes and sends them to a mobile device, which in turn sends them to a first computing server, where cardiac biopotential signals are decoded, reconstructed into an ECG, labeled and stored. The biopotential signals are then sent at the same time to a second computing server and to a third computing server, which in turn deliver a series of potential scenarios.

### TECHNICAL FIELD

This invention is situated in the medical device field, presenting a biopotential signal collection and processing system for cardiac monitoring, prediction and alerts, as well as methods or procedures to acquire data by means of a biometric data acquisition device to then send such biometric data to a remote or “cloud” server (which represent an electrocardiogram or ECG), and also methods for heart disease detection are presented. Thus, this invention presents an alternative for individuals with any cardiac issues and/or any individuals who want to monitor their heart, collecting the data required to create an electrocardiogram (ECG) using one or several cardiac data acquisition devices with electronic connection to a portable device such as a cell phone, tablet or computer, where collected data are grouped, sent to the cloud and analyzed by means of a neural network algorithm, in order to monitor the user's heart, anticipate a cardiac episode and issue an alert if the ECG yields an anomaly, among other functions or actions that may save the user's life.

Neural networks consist of two or more neurons (X1, X2, . . . . Xn) that try imitating the functioning of human brain; a neuron in the context of this invention is an abstract object that receives several incoming data (numeric), which are assigned a weight (W1, W2, . . . . Wn), does a weighted addition Ui=Σj(WijYj), and this addition is standardized through a non-linear mathematical function, such as ReLu, sigmoid, among others; the result after standardization is taken as the output. Neural networks are normally comprised of one or more layers (Y1, Y2, . . . . Yj); in this context, a layer refers to a grouped neuron number, where all of them receive the same inputs (in a fully connected network), but where each neuron has different weights; thus, the first layer is referred to as the input layer, intermediate layers are referred to as hidden layers, and the last layer is referred to as the output layer. These neuron layers may be sequential, i.e., the outputs of a neuron layer are received as inputs in the next layer, where each layer may have a different number of neurons. Neural networks normally contain three types of databases. The first type of database is the training one, which is intended to adjust the weight (W) of each neuron; the second type of database is the validation database, which is used to validate weights (W) and to eliminate the overfitting issue; the third type of database is the testing one, which is very important because it allows us to measure the error and know how well-trained the neural network is.

In the particular case of this invention, the neural network is initially trained under an electrocardiogram dataset. This set may be a public database, data of the user over time or a database of heart diseases obtained from different sources, the purpose of which is to train the neural network for it to recognize patterns in an ECG in order to be able to detect or predict heart diseases. To achieve this, the neural network used by this invention has been trained under the backpropagation theory, i.e., the values of the input layer of the neural network are assigned weights with randomly established values. The data of the input layer are a series of data describing an ECG the attribute of which is a clinical profile (i.e., it is known beforehand if the ECG includes a heart disease, the type of heart disease, or if it is a “normal” ECG). In the end, the output value of the neural network is compared with the actual output (the known clinical profile) by means of an error function and, based on this, weights are changed. This is reiterated all the time, being the way in which the neural network itself “learns” or, in other words, is adapted to the data of the user over time, also leaving the door open to admit in the database new data on heart diseases. The last layer of the neural network of this invention consists of a single neuron that grants a value through which, when compared to values obtained from laboratory testing, it can be determined if the examined ECG reflects any heart disease or if the state of the user's heart is “normal. In the former case, an alarm system is activated; otherwise, the “normal” label is kept in the user's portable device.

### Prior Technique

### Background

We used to read in science fiction books about various gadgets that were able to acquire biometric signals with which the life of characters would be saved; or even in movies or television, we would yearn for space travels wearing space suits remotely monitoring our heart rate, breathing, organ functions and so forth. At that time, it seemed to be a distant future, but now we are astonished with affordable wristwatches that can measure our heart rate, count our daily steps, calculate calories when exercising, among other features. Data can even be exported to a computer for future analysis using specialized software or apps. Within the scope of the invention in question, we can mention some examples of medical devices that require data to be analyzed with a view to alert or assist medical staff in treating heart diseases, including:

Document WO20211866 by Louise Rydén, regarding a device using multiple sensors arranged in clothing, with such sensors collecting data to generate an ECG. Sensors communicate wirelessly to a first master driver, which collects information from sensors, saves, process and sends such information to a control computer, which is configured to detect anomalies in the ECG. If an anomaly is detected, the control computer issues an alarm through an indicator.

Document US20200214618 by Rik Vullings, which describes and illustrates a device that obtains ECG signals from a fetus. The device has multiple sensors that are arranged on the abdomen of the mother. Devices send the information to a master control, which collects and stores information from the sensors, and it also processes such information, first generating an ECG vector. Such neonate's ECG vector is processed by means of an already trained machine learning process that allows classifying the baby's ECG. Data are also used as input for the machine learning process.

Document US20200205745 by Ali Khosousi et al., which illustrates and describes a method to detect anomalies and biophysical signals. In order to achieve this, biophysical signals area acquired in one or several channels in a computer. Such biophysical signals are pre-processed to generate datasets, each of which contains a full cardiac cycle. Datasets are processed, yielding as a result a value that indicates the presence of a heart disease. Datasets are processed using deep neural networks, which are trained using biophysical signal data acquired from patients with specific heart diseases and labeled with the presence or absence of a heart disease. The result can be yielded as a report or displayed on the computer screen.

Document U.S. Pat. No. 7,194,298 by Louis Massicotte et al., which describes a method to determine a trend in the ECG signal, where the method learns based on information that is acquired little by little from the user. The method consists in extracting baseline points from said ECG signal; each baseline point is compared with default thresholds by means of a slow adaptive learning system that uses the data of the user. Such thresholds have vertical and horizontal limits in the ECG graph, and those limits are modified over time. If baseline points are within the threshold, then an analysis is run to look into unlikely points. If points are determinable, then a trend in the ECG signal monitoring can be determined based on said new determinable points.

The examination of the aforementioned documents evinces the need of a cardiac monitoring system that is not intrusive for patients, with a convenient size, easily manufactured, low cost, with few maintenance requirements, long duration battery, reliable, with which, if necessary, several devices arranged on the user can be used in order to obtain different channels or leads to generate an ECG and, based on that ECG, it can be determined if the user is in optimal condition or if the patient is experiencing a cardiac event, which allows alerting the user and medical team; purposes that this invention intends to thoroughly cover.

### BRIEF DESCRIPTION OF THE INVENTION

The biopotential signal collection and processing system for cardiac monitoring, prediction and alerts described below consists in a biopotential data acquisition device fitted with a wireless module electronically connected to a battery and at least one biopotential sensor. The aforementioned sensor collects cardiac biopotential signals (ECG) that are delivered to the wireless module, which adjusts, codes and sends them to a mobile device, which in turn sends them to a first computing server, where cardiac biopotential signals are decoded, reconstructed into an ECG, labeled and stored. Afterwards, such biopotential signals are sent at the same time to a second computing server and to a third computing server. The second computing server hosts a medical platform that allows consulting data, as well as alarm recording. The third computing server processes the biopotential signals by means of neural networks; a first neural network determines if the user's status is “normal” or if there is a heart disease; if a heart disease is detected, a second neural network compares the biopotential signal against preestablished heart disease signals that can be detected through cardiac leads, delivering a series of potential scenarios. Once the statistical mode of these scenarios has been analyzed, the probability of occurrence is determined. If the user's cardiac status is not in order, an alert is sent to the user and/or authorized interested person; if the user's cardiac status is in order, the user and/or authorized interested person are notified. In both cases, the labeled ECG is saved to be used in the neural network learning module.

### BRIEF DESCRIPTION OF FIGURES

FIG. 1 shows an electric block diagram of the biopotential signal acquisition device.

FIGS. 2, 3, 4, 5, 6, 7, 8, 9, 10 show a block diagram of the operation, functioning and signals of the biopotential signal acquisition device, which for readability is illustrated in nine pages.

FIG. 11 shows a flow diagram of the operation of the biopotential signal collection and processing system for cardiac monitoring, prediction and alerts.

FIG. 12 shows a block diagram illustrating the cardiac biopotential signal processing.

FIG. 13 shows a diagram illustrating the three layers of a neural network.

FIG. 14 shows a diagram of the biopotential signal acquisition device.

FIG. 15 illustrates a diagram with a user wearing a biopotential signal acquisition device.

FIG. 16 illustrates a diagram with a user wearing multiple biopotential signal acquisition devices.

FIGS. 17a, 17b, 17c illustrate different screens of the mobile app being implemented in the mobile device or computer.

FIG. 18 shows a diagram of an input neuron layer with weights and activation function.

FIG. 19 shows a diagram of the intermediate layer of a neural network.

FIG. 20 shows a diagram of the intermediate and output layers of the first neural network.

FIG. 21 shows a diagram of the intermediate and output layers of the second neural network.

### DETAIL DESCRIPTION OF THE INVENTION

Alterations of the structure described herein may be intended by those knowledgeable about the matter. Nevertheless, it is necessary to understand that this description is related to the preferred modalities of the invention, only for illustration purposes, and must not be understood as limitative for the invention.

When reference is made to any structural characteristic, mechanical link or component, the following terms: secured by a mechanical means, mechanically secured, mechanically attached, mechanically fastened or mechanically connected, it must be understood that any means known in the technique may be used to mechanically join, for example: screws and nuts with or without washers, rivets, screws in threaded holes, welding of any sort (arc, tig, mig, etc.), friction welding, ultrasound welding, glues, binders, overmolding, inserts, pressing, inlaying, interference assembly, snaps, flanges and belts, wedges, bolts, setscrews, grooves and lock washers, which can be used alone or together, among other well-known items of this kind.

Other aspects, modalities and advantages of those example aspects and modalities are discussed further below. The description provides illustrative examples of various aspects and modalities of this invention, and intends to provide an overview or framework to understand the nature and characteristics of the claim aspects and modalities. The accompanying figures are included to provide additional illustration and understanding of the different aspects and modalities, and are incorporated and part of this specification. Figures, together with the description, are intended to explain the described and claimed aspects and modalities.

The following definitions are provided for a better understanding of the invention:

Mechanically coupled—This refers to the transmission of mechanical power from one item to another one by means of gears, friction pulleys, band pulleys, magnetic means, mechanical transmissions, arrows, mechanical couplings, dynamic couplings, universal gaskets, Hook gaskets, constant-velocity joints, cardan joints, wet means, among others.

Electronically coupled, electronically connected—This refers to wire or wireless connection between electronic items e.g.: sensor, driver, actuator, microcontroller, microprocessor, antenna, peripheral circuit, etc., in order to receive/send an analog or digital signal.

Electrically connected, electrically coupled—This refers to the power or electric energy connection or supply from a power source to electric or electronic items that require being energized in order to be able to operate.

Approximately—The use of this term provides a given additional range relative to the numeric value to which it is being applied. Said additional range is ±10%. By way of illustration and not limitation, if it is said “approximately 40 cm”, the exact range described and/or claimed ranges between 36 cm and 44 cm.

Multiple—Two or more items of a same species; multitude, large number of some things, or the higher number of them.

ECG—Electrocardiogram.

Server—A short term used in computing that refers to or is understood as a computing server.

Data to be processed module—A database where the ECG signals to be processed by neural networks are backed up, where such signals have been duly packaged, digitized, conditioned, rebuilt and labeled.

Reference data module—Database containing packaged, digitized, conditioned, rebuilt and labeled ECG signals known to be related to a known heart disease, and user's “normal” ECG data both at rest or under ordinary cardiac activity, or in a medium cardiac activity status, high cardiac activity status or extraordinary cardiac activity status.

Machine learning module for the calibration of the classification module—Database where digitized, conditioned, rebuilt and labeled ECG data related to a heart disease are backed up, either if such data come from an external database or from heart diseases detected by the second neural network. This module also contains the ECG data of “Normal” or “Extraordinary” user's status at rest or under ordinary cardiac activity, in a medium cardiac activity status, high cardiac activity status or extraordinary cardiac activity status, with such data used to regularly recalibrate the weights “W” of each neural network for update and continuous improvement.

“Normal” user's status—This means that the user's ECG is within parameters deemed “normal”.

“Extraordinary” user's status—This means that the user's ECG is outside parameters and may represent a heart disease.

User's reference-adjusted model—This refers to the update of the databases of neural network modules with data acquired from the user over time, which allows regularly adjusting the weights “W” of each neural network.

Medical platform—An app or software that allows backing up, displaying and consulting digitized, conditioned, rebuilt and labeled ECG signals from the BSAD 10; it is also in charge of issuing alarm indications both on the screen of the medical platform itself and sending the alarm indication through the server 11 to the mobile device or computer 20.

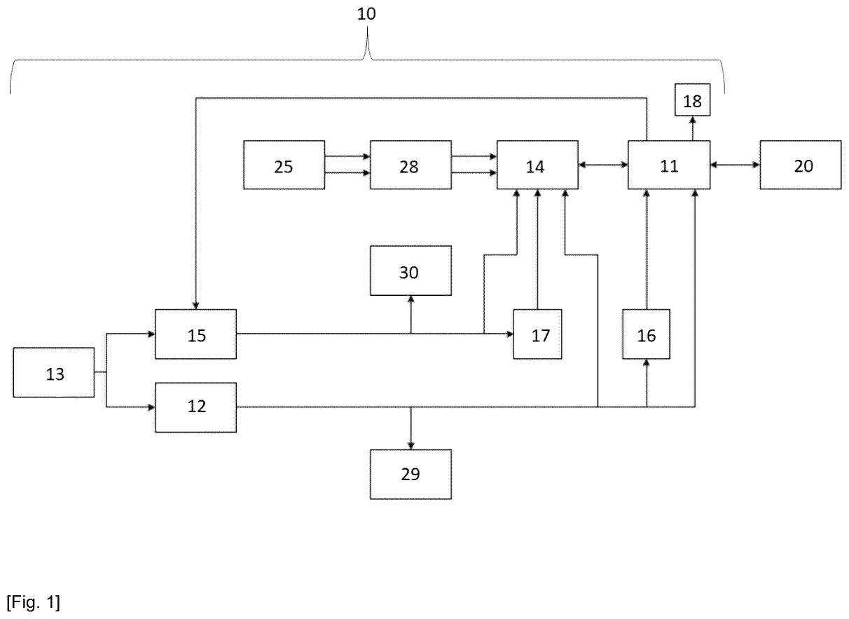

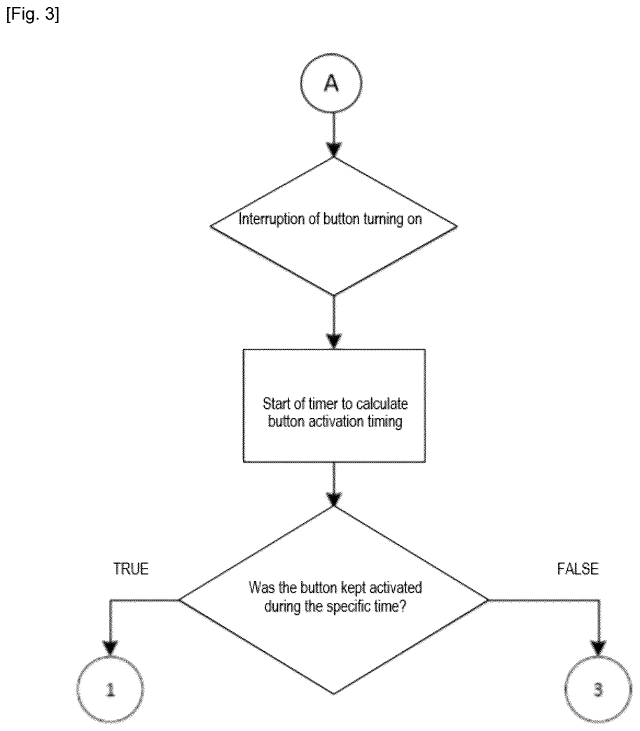

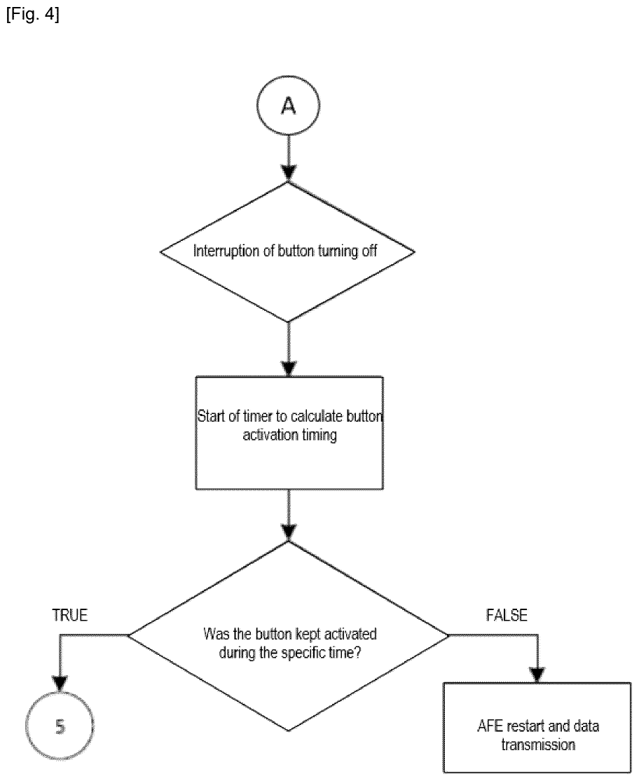

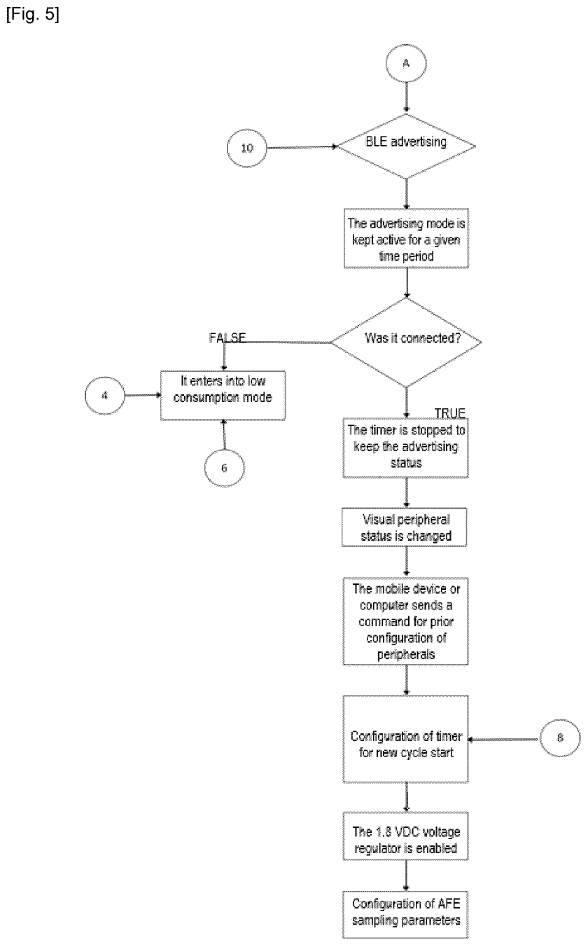

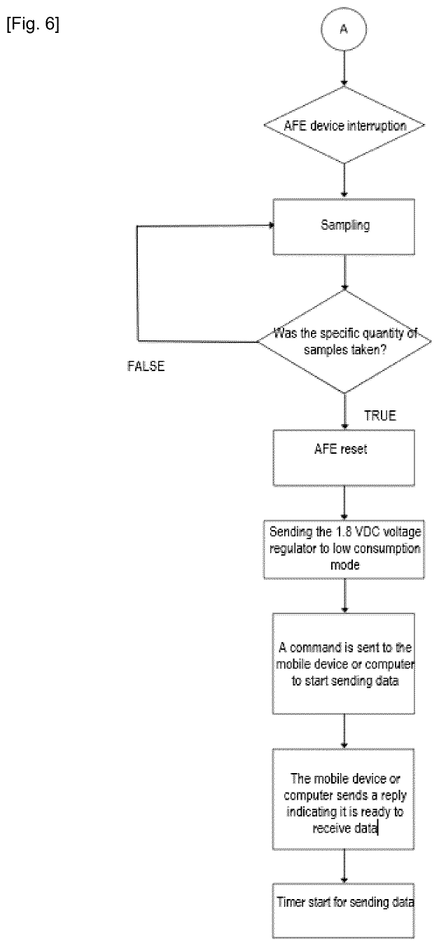

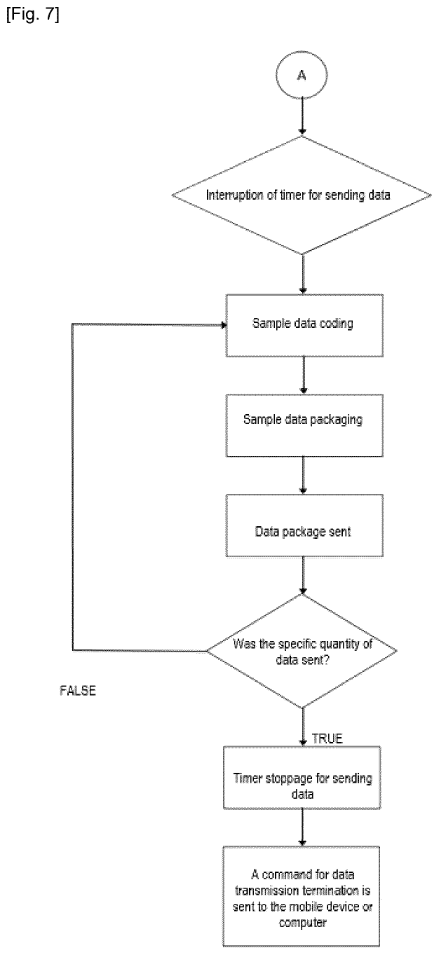

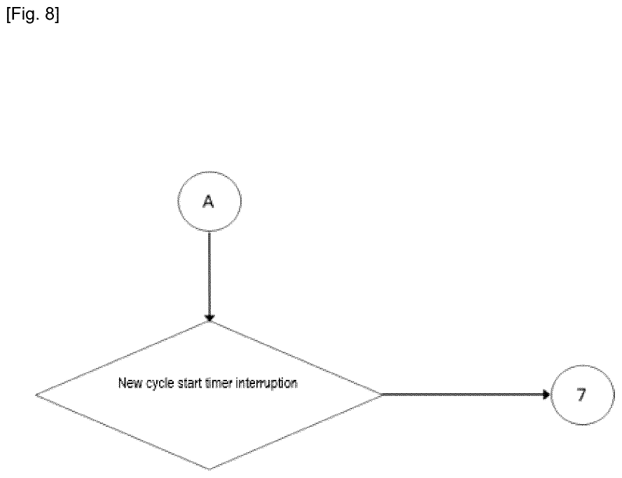

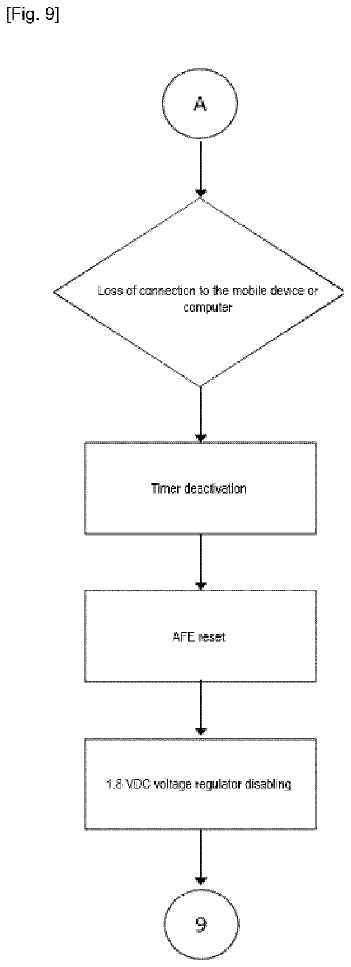

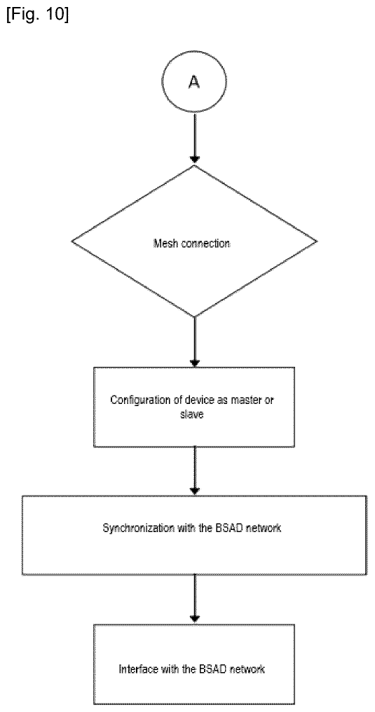

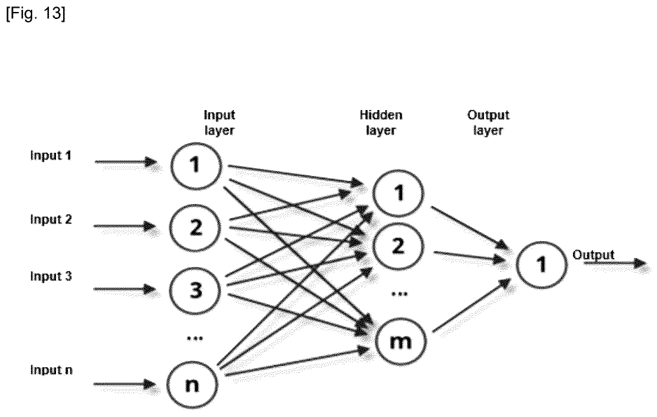

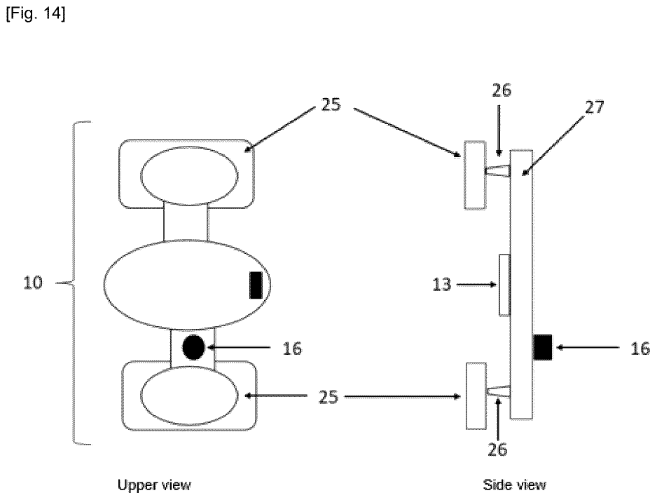

FIGS. 1 to 22 are indistinctly used in the following description:

As illustrated in FIG. 1, the biopotential signal acquisition device or BSAD 10, is comprised of the wireless module 11, which includes a microcontroller and an ultra-low consumption antenna at 2.4 GHz. Likewise, the BSAD 10 includes a first 2.4 V voltage regulator 12, in addition to a 3 V battery 13 that supplies both the microcontroller and the antenna, and a second regulator 15. The wireless module 11 receives the signal from an Analog Front End (AFE) digital block (14), which in a preferred modality is provided with an analog-to-digital converter (ADC) and at least one biopotential sensor, which acquires the biopotential signals through at least one electrode 25 electrically connected to a low-pass filter 28 in order for the ADC to adjust the signal from at least one biopotential sensor, so that it can be subsequently processed by the wireless module 11, In a preferred modality, the battery 13 is electrically coupled with the first 2.4 V voltage regulator 12, which supplies the wireless module 11, the button 16 and the AFE digital block 14. In parallel to the process described above, a second 1.8 V voltage regulator 15 supplies a first bypass capacitor module, the AFE digital module 30 and an oscillator 17 electrically connected to the AFE digital module 14. The 1.8 V voltage regulator 15 is controlled by a digital signal issued by the wireless module 11. In this preferred modality, the first 2.4 V voltage regulator 12 supplies a first bypass capacitor bank 29, the wireless module 11 and the button 16; as evident in an alternative modality, the wireless module 11 may be electrically coupled with the first 2.4 V voltage regulator 12 or the battery 13, omitting the use of the first voltage regulator 12. In any case for the alternate modality, the wireless module 11 is electronically coupled with the AFE 14, the second 1.8 V voltage regulator 15, the button 16 and the light-emitting diode (LED) 18 and, if applicable, an ADC. In an alternative modality, a BSAD 10 can have multiple AFEs 14. In an alternative modality, the signal of at least one AFE 14 is directly received by the wireless module 11, which is an analog signal, reason why the wireless module 11 itself must have an internal ADC with which it can convert or “translate” the analog signal into a digital signal. In an alternative modality, the AFE 14 signal reaches an ADC, which converts or “translates” the analog signal obtained from the user, a digital signal that is received and subsequently processed by the wireless module 11.

A low-consumption antenna is embedded in the wireless module 11, which is used to send and receive information. In a preferred modality, the antenna is Bluetooth compatible. In an alternative modality, any other protocol or technology available on the market can be used, including, without limitation: Zigbee, Thread, WIFI, XBee, ZWave, Symphony Link, Sigfox, radio frequency, LoRa, among others. By means of an antenna inside the wireless module (11), communication is established with the mobile device or computer 20; the antenna allows sending and receiving data to and from the wireless module (11), from and to the mobile device or computer 20, and receiving service and diagnostic indications, programming or connections from the BSAD 10 itself. In a preferred modality, the wireless module 11 sends and receives information for certain time periods, e.g., when activated to send information to the mobile device or computer 20 every 5 minutes or, once such period has elapsed, it can enter into a low-energy consumption mode for a 3-minute period. A first alternative modality is presented when the wireless module 11 “awakens” every time it requires sending or receiving information. A second alternative modality is presented when the wireless module 11 includes a low-energy consumption type antenna, which allows the wireless module 11 to be “on” all the time, and receive or send information as required by the wireless module 11.

In a third alternative modality referred to as preferred, there are multiple BSAD 10 arranged on several body parts of the user in order to collect multiple cardiac biopotential signals, which can be processed in different channels, i.e., one channel per BSAD 10. In this case, there is a BSAD 10 that will serve as “master”, which will coordinate the rest of BSADs 10, which will serve as “slaves”, collecting the information package of each BSAD, following a communication protocol that prevents the collapse of signals, e.g., assign a number to each BSAD 10 of the network and name them per order every given time period, e.g., when the “master” BSAD 10 names the BSAD 10 “slave 1”, which will transmit for a given time period, such as 10 s, and then will terminate communication to continue with the “slave 2” BSAD 10, which will also transmit to the “master” BSAD 10 for a given time period and so forth until the “slave n” BSAD 10 is reached. Subsequently, the involved BSADs 10 enter into a low consumption mode for a given time period. Once the time period has elapsed, all BSADs 10 collect a new sample, resuming the cycle once again.

In any modality, the digital signal is coded by the wireless module 11, which helps avoiding interference with other pieces of equipment, adding security when data are sent to the mobile device or computer 18, and this also helps packaging data and reducing transmission times, which contributes to extend the lifespan of the battery 13. The coding media that can be used are well known in the field, and they include the following: Numeric, alphabetic and alphanumeric coding, which can be indistinctly used in this invention.

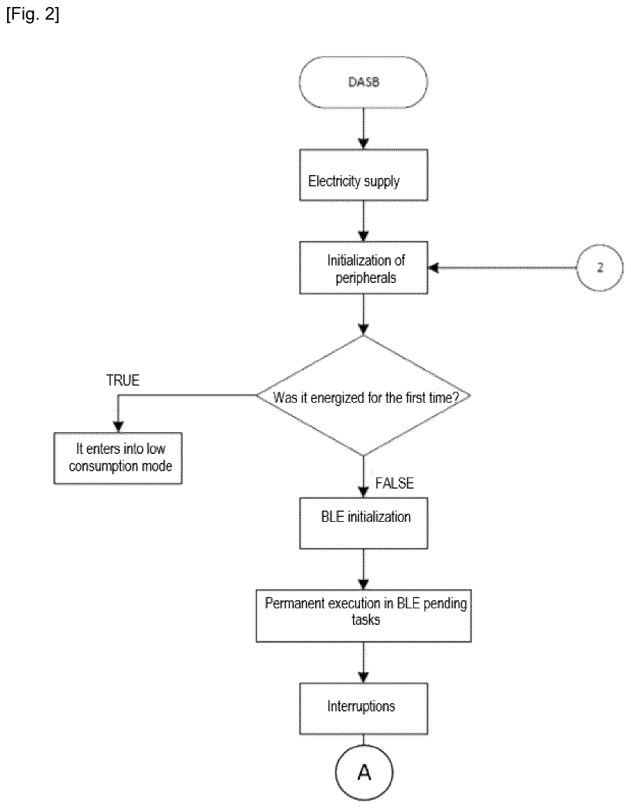

In FIGS. 2 to 10, we can see the signal methods or processes carried out by a BSAD 10. Particularly, such methods must be considered here as if inserted verbatim. In a preferred modality, the first block represents the electric supply, such as a battery 13, that allows running peripherals. If the BSAD 10 was energized for the first time, it enters a low consumption mode. In order to run or “awaken” the wireless module 11, the user must keep activated the button 16 for a specific time (e.g., 3 seconds), which will allow the BSAD 10 to return to the start of peripherals where, otherwise, if the BSAD 10 was not energized for the first time, the Bluetooth Low Energy (BLE) module with which the antenna is fitted in this preferred modality will be started, and then the BLE pending tasks will be permanently implemented. Afterwards, the wireless module 11 activates the antenna, which in turn puts the BLE in advertising mode (this means the mobile device or computer 20 can establish a connection to the BSAD 10) for a period of time (e.g., 1 min). If a connection to the BSAD 10 was not made, it enters a low consumption mode for a period of time (e.g., 10 ms to 5 min), where, otherwise, if the connection was correctly established, the timer will be stopped in order to keep the connection status. Then, the status of visual peripherals, in this case LED 18, is changed; therefore, the mobile device or computer 20 sends an indication for prior configuration of peripherals. Subsequently, a timer configuration is carried out to start the new cycle and then the second 1.8 VDC voltage regulator 15 is enabled in order to carry out a configuration of the sampling parameters of the AFE device 14.

Interruption of the AFE device 14 indicates that the sampling or biopotential signal acquisition has been started, i.e., that biopotential sensors send the acquired signals to the ADC or directly to the wireless module 11 (depending on the modality), where, in any case, the wireless module 11 acquires a specific quantity of samples or signals (called data). Once this has happened, the AFE device 14 is reset. Then, the second 1.8 VDC voltage regulator is sent to a low consumption mode. Once this has been done, the wireless module 11 sends an indication to the mobile device or computer 20 in order to start sending data, where the mobile device or computer 20 “responds” to the wireless module 11 that it is prepared for data receipt, starting the timer for the sending of data.

When sending data is required, the wireless module 11 interrupts the timer to carry out the coding and packaging of data prior to sending data to the mobile device or computer 20, or to a “master” BSAD 10. In any case, during interruption of the timer (and prior to data being sent by the wireless module 11), the data sampled by the BSAD 10 are coded. Then, the BSAD 10 packages the sampled data in order to subsequently send the data package through the wireless module 11, which in turn ends the interruption of the timer, allowing it to continue counting, which is performed until the specific quantity of coded and packaged data to be sent is reached. Once the mobile device or computer 20 has received the specific quantity of coded and packaged data (i.e., it has the complete data), it sends a signal to the wireless module 11 in order for it to stop the timer for sending data. Finally, the wireless module 11 sends a data transmission termination indication to the mobile device or computer 20.

Interruption of the new cycle start timer specifically returns at the timer configuration section for the start of the new cycle as described above.

If connection to the mobile device or computer 20 is lost, timers are deactivated, the AFE 14 is traced, and the second 1.8 VDC voltage is disabled, which causes a return to the BLE advertising process, where all of its cycle, which has already been described above, is repeated.

Button 16 shutdown interruption may happen at any time, and when activated, this starts the timer in order to calculate the timing for activation of the button 16. If the button 16 was kept activated for the specific time (e.g., 5 s) a low consumption mode is applied. On the other hand, if the button 16 was not kept activated for the specific time, the AFE device (14) and data transmission are restarted.

Assuming an alternative modality, the BSAD 10 can perform a mesh connection. First, a BSAD 10 is configured as “master” or “slave” in order to continue a synchronization with the BSAD 10 network, and in this way an interface with the BSAD 10 network is achieved.

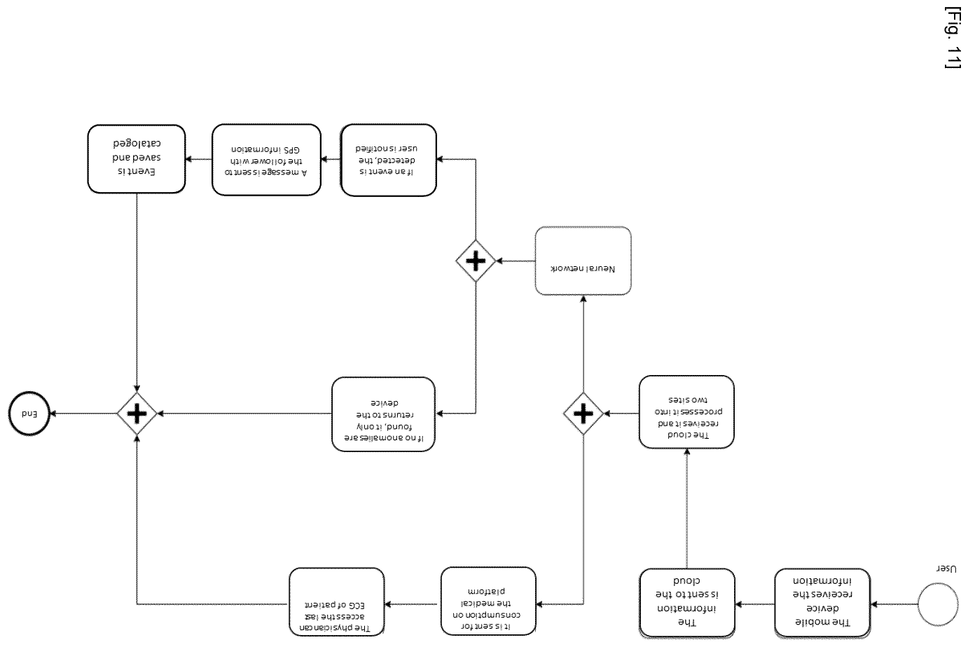

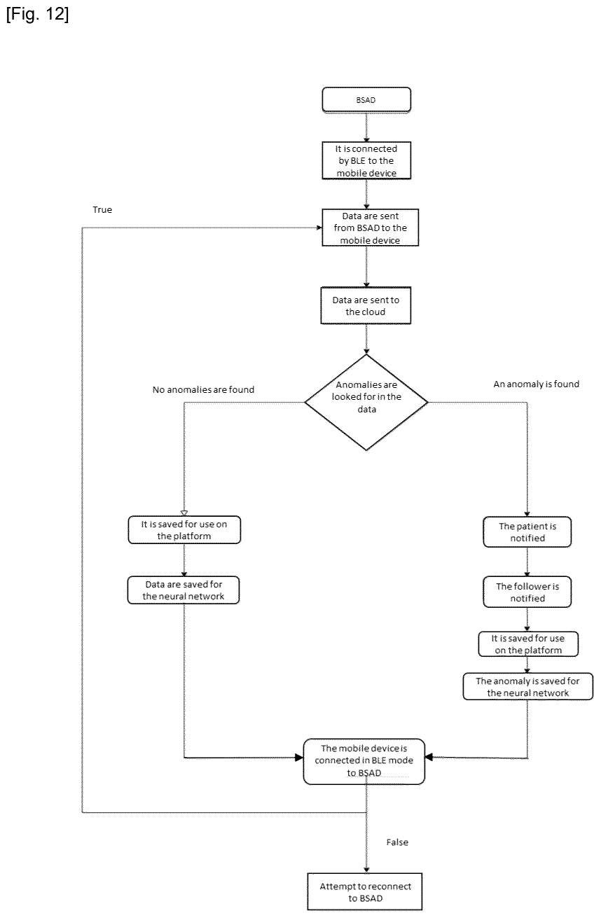

As illustrated in FIG. 11, once the coded and packaged data have been received by the mobile device or computer 20, it in turn transmits the data to a computing server or first server 21 by WiFi or any other means that grant it internet access (illustrated as a “cloud” in FIG. 12). When the mobile device or computer 20 is in possession of the server 21, it decodes and rebuilds the conditioned digital signal, and duly labels it for identification and traceability, as well as subsequent storage. The aforementioned label includes as a minimum a number, lot or consecutive number of the biopotential signal sample package, user's identification number, which can be associated with a database holding personal data such as name, address, mobile telephone number, emergency telephone number, physician's telephone number, physician's name, among other data. Then, the digitized, conditioned, labeled and rebuilt signal package is sent to a second server 22 that supports a medical platform with software or apps dedicated to users and authorized followers, where such digitized, conditioned and rebuilt signal package is stored and can be consulted on the aforementioned platform. Simultaneously, the digitized, conditioned, labeled and rebuilt signal package is sent to a third processing server 23, which processes and classifies the digitized, conditioned, labeled and rebuilt signal package in search of heart diseases. In a preferred modality (illustrated in FIG. 11), the search of heart diseases is based on the use of a couple of tandem neural networks (i.e., a first neural network is used and, if a heart disease is detected, the second neural network is used to identify the potential heart disease, as well as its percentage of affinity with the study ECG), where each of said neural networks has a data to be processed module, a reference data module and a machine learning module for calibration of the classification model, which results in a user-adjusted model. Then, aforementioned digitized, conditioned, labeled and rebuilt signal package is processed and classified using the user's reference-adjusted model in any of the following statuses: At rest or under ordinary cardiac activity, and in a medium, high or extraordinary cardiac activity status. The foregoing is intended to determine the user's cardiac activity status based on the acquired digitized, conditioned, labeled and rebuilt signal package. Once the cardiac activity status which the discussed digitized, conditioned and rebuilt signal package belongs to has been determined, the affinity label of the user's cardiac activity is added. With such data, the digitized, conditioned, labeled and rebuilt signal package is stored in the machine learning module for calibration of the classification model with which the user-adjusted neural network model is recalibrated. This is carried out by recalculating the weights “W” of both neural networks based on the data obtained from digitized, conditioned, labeled and rebuilt signals that are regularly supplied to or saved in the aforementioned machine learning module for calibration of the classification model, which leads to a user's “ordinary” or “extraordinary” status classification by the first neural network, with the framing with a heart disease profile recorded in the database of the reference data module (tachycardia, bradycardia, arrhythmia, etc.) resulting from the second neural network. If an extraordinary status if found when a heart disease is framed, authorized followers are notified by means of an alert issued by the second server 22, which is received by the mobile device 20 through the first server 21, projecting the cardiac status extracted from the labeled digitized, conditioned and rebuilt signal package. Besides, such data are saved in the machine learning module for calibration of the classification model.

In an alternative modality (not illustrated), there are two stages for the search of heart diseases. The first stage for the search of heart diseases is based on the sinus rhythm, while the second stage is based on the tandem use of two neural networks. Thus, the first stage, which is based on the sinus rhythm, is performed when a probability of a cardiac event is estimated for the package of already cataloged digitized, conditioned and rebuilt signals upon analysis of the digitized, conditioned and rebuilt signal package during the search of heart diseases that can be analyzed or that are evident just by the determination of the sinus rhythm (tachycardia, bradycardia, arrhythmia).

If from the foregoing step it is determined that the sinus rhythm included in the digitized, conditioned and rebuilt signal package has high affinity with a sinus rhythm-related heart disease, the server 23 proceeds to perform a new comparison, where the digitized, conditioned, labeled and rebuilt signal package is now compared with the heart disease models stored in the database of the third server 23, with which a percentage of affinity with a heart disease model is determined. If high affinity is determined (affinity percentage over 60%) with any of the stored heart disease models, an alert is issued to the server 22, which sends the alert to the mobile device 20 through the server 21, showing the rebuilt ECG that has been extracted from the digitized, conditioned and rebuilt signal package labeled with the affinity percentage, as well as the type of heart disease in question, and also a notification is sent to the user's mobile device or computer 20. If it is determined that there is no high affinity with any of the heart disease models stored in the digitized, conditioned and rebuilt signal package, a new label is added indicating that this second step of heart disease comparison has been performed, backing up again the digitized, conditioned and rebuilt signal package in the data to be processed module of the neural network.

Continuing with the alternative modality (not illustrated), the search of heart diseases based on a couple of tandem neural networks is performed when the package of digitized, conditioned, rebuilt and labeled signals that have been subject to the first stage for the search of heart diseases based on the sinus rhythm in the server 23 is backed up in the data to be processed module, with which signals are appropriate to be processed now in the first neural network, which enables us to know if there is an “ordinary” or “extraordinary” user's event or status. In the case of an “extraordinary” event or status, the second neural network, which enables us to know the heart disease with higher affinity and its affinity percentage, is used.

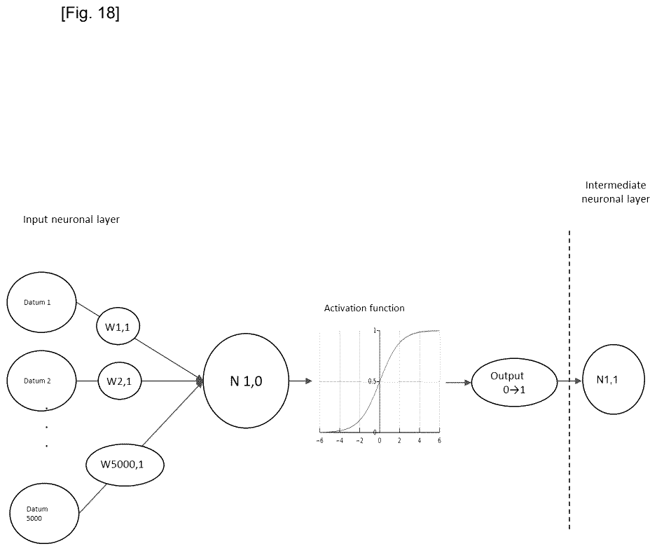

In any modality, the search of heart diseases based on a couple of tandem neural networks is carried out when the discussed digitized, conditioned and rebuilt signals are deposited in the data to be processed module, from where they are taken to pass them through the first neural network, which is duly trained and calibrated, i.e., the weights “W” for each of the neurons that comprise the input layer and the intermediate layers (which is detailed in the example) have been obtained, as illustrated in FIGS. 18, 19 and 20. For the first neural network in its preferred modality, the input neuron layer hosts a piece of data of the package of digitized, rebuilt and labeled signals that have been subject to the two aforementioned heart disease comparison processes, being evident that the number of neurons of the input layer depends on the number of data included in such signal package, typically between 100 and 10,000 data, which depends to some extent on the storage capacity, type of battery, antenna transmission rate, data transmission protocol, distance to the data receiver, among other technical features of the BSAD 10. In a preferred modality, the first neural network is comprised of a couple of intermediate neuron layers, where the number of neurons of the first intermediate neuron layer is between 30% and 50% the number of neurons of the input layer. On its part, the second intermediate neuron layer has between 3% and 20% the number of neurons of the first intermediate neuron layer, with which the number of neurons per layer is decreased. In an alternative modality, the first neural network may be comprised of a single intermediate neuron layer, where the number of neurons of the single intermediate neuron layer is between 10% and 50% the number of neurons of the input layer. In other modalities, the first neural network may have three or more intermediate neuron layers, which will allow improving its performance and reliability, but a more powerful computer equipment will be required, and response times will be longer due to the high amount of data and operations that the computer should carry out. Picking up the thread, in any modality of the first neural network, the output neuron layer is comprised of a single neuron, which evaluates the possibility of having a heart disease. Thus, if the number yielded by the output of the neuron of the output layer of the first neuron is close to zero (e.g., values from zero to 0.69), this means that the package of digitized, rebuilt and labeled signals that have been subject to the two aforementioned heart disease comparison processes has a low or null correlation with a heart disease, i.e., it is in a user's “ordinary” status, to which the server 23, in a preferred modality, sends a couple of simultaneous “everything normal” or “nothing to be reported” signals to the second server 22, so the latter records it on the medical platform, and in the mobile device or computer through the server 21. In an alternative modality, the server 23 sends an “everything normal” or “nothing to be reported” signal to the server 22, so it records it on the medical platform. In any modality, the package of digitized signals that were supplied to the first neural network is saved in the machine learning module for calibration of the classification model, which will help to have more data to subsequently calibrate the weights “W” of the first neural network, which will in turn help it to have better precision with the use of the system that is the subject matter of this invention. In the opposite case, i.e., if the number yielded by the output of the neuron of the output layer of the first neural network is close to 1 (e.g., values between 0.7 and 1), this means that the package of digitized, rebuilt and labeled signals that have been subject has high correlation with a heart disease, i.e., it is in a user's “extraordinary” status and, therefore, it is necessary to use the second neural network to find the heart disease with the best correlation with said digitized signal package supplied to the first neural network. Thus, such digitized signal package is now transferred to the data to be processed module of the second neural network.

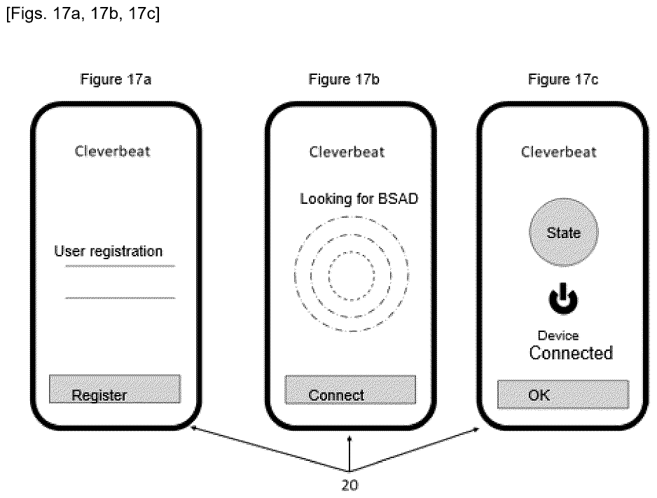

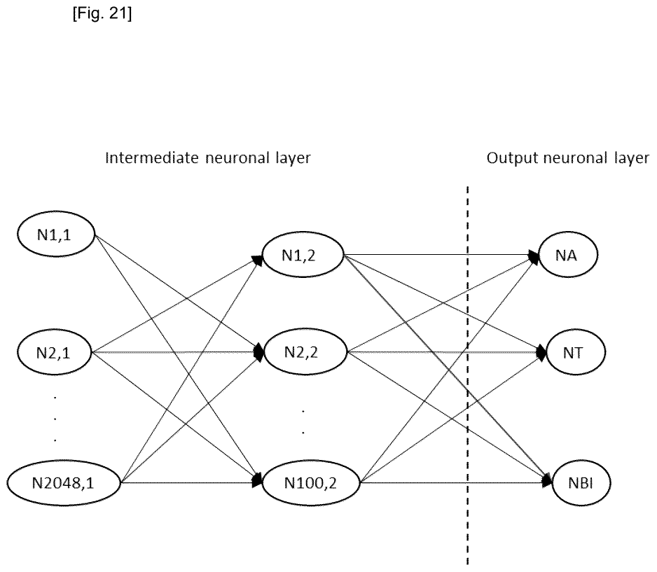

The second neural network is comprised of a database of weights “W” somewhat different from the first neural network studied above, in addition to including multiple output neurons; however, it uses a similar schema for the input neuron layer and for intermediate layers. In its preferred modality, the input neuron layer hosts a piece of data of the package of the aforementioned digitized, rebuilt and labeled signals that are now backed up in the data to be processed module of the second neural network, remembering that such digitized signals have been processed by the first neural network. As in the first neural network, digitized signals located in the data to be processed module are processed. It is also worth mentioning that the number of neurons of the input layer of the second neural network depends on the number of data included in said signal package. As already noted above, these typically include between 100 and 10,000 data, which depends to some extent on the storage capacity, type of battery, antenna transmission rate, data transmission protocol, distance to the data receiver, among other technical features of the BSAD 10. Similarly to the first neural network, the discussed second neural network in a preferred modality includes a couple of intermediate neuron layers, where the number of neurons of the first intermediate neuron layer is between 30% and 50% the number of neurons of the input layer. On its part, the second intermediate neuron layer has between 3% and 20% the number of neurons of the first intermediate neuron layer, with which the number of neurons per layer is decreased. In an alternative modality, the second neural network may be comprised of a single intermediate neuron layer, where the number of neurons of the single intermediate neuron layer is between 10% and 50% the number of neurons of the input layer. In other modalities, the second neural network may have three or more intermediate neuron layers, which will allow improving its performance and reliability, but a more powerful computer equipment will be required, and response times will be longer due to the high amount of data and operations that the computer should carry out. Picking up the thread, in any modality of the second neural network, the output neuron layer is comprised of multiple neurons, where each of the neurons of the output layer is related to a heart disease (see FIG. 21), being clear that the number of neurons of the output layer of the aforementioned second neural network is equal to the number of heart diseases that have been studied and that have defined affinities, so that the output of a given neuron of the output layer of the second neural network will indicate the probability that the user has a given heart disease (e.g., arrhythmia) and its connections with data indicating such heart disease will have much greater weights “W”, which is the case for all supported diseases. This information is processed by statistical means and generates a potential scenario that indicates the most related heart disease or heart diseases, as well as their affinity percentage. Using this information, the heart disease with the highest affinity among potential heart diseases is identified by statistical means. Once such heart disease has been identified, if it has a high affinity percentage (e.g., 60% and higher), the user and authorized followers are warned or alerted about the type of scenario with which affinity has been found (the type of heart disease and its affinity percentage). This is achieved by sending such information to the second server 22. Such information is also sent to the first server 21 for storage in the machine learning module for calibration of the classification model of both neural networks. On its part, the server 22 processes the information and issues an alert, which is deployed on the medical platform, notifying the individuals authorized for emergencies, and also being sent to the first server 21, which in turn forwards the alert to the mobile device or computer 20; the latter will display on screen an alert signal, as well as a button that allows knowing that the user has seen the alert (see FIG. 17c). In an alternative modality, it will also be possible to deploy a button indicating if the user is well.

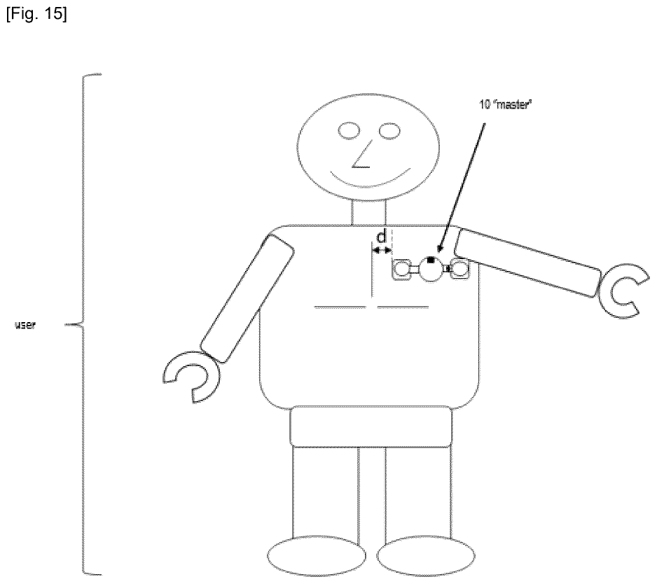

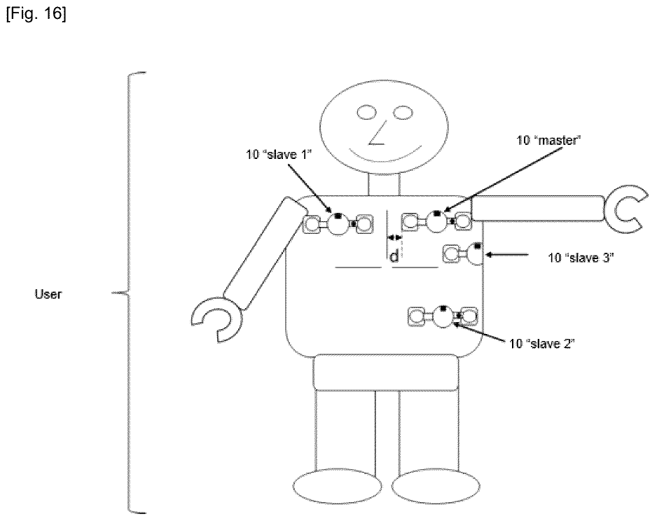

We will use an example that will help us to describe in an educational manner the biopotential signal collection and processing system for cardiac monitoring, prediction and alerts that, as noted above, uses a couple of tandem neural networks (first a neural network is used to verify whether there is a heart disease; if that is the case, a second neural network is used to detect the type of heart disease), and the example is also very useful in order to reinforce the understanding of the method for the acquisition and sending of biopotential data (ECG) through a BSAD 10 or multiple BSADs. The example in question also helps us to describe the method for detection of heart diseases based on a couple of neural networks subject matter of this invention. Thus, we have that, as illustrated in FIG. 16, there is a user, who wears a BSAD 10 arranged on the left side of the chest, at a distance “d” from the central portion where both chest sides join (see FIG. 15). Distance “d” is measured using the three middle fingers (i.e., index, middle and ring). An alternative modality is shown in FIG. 16, where a first “master” BSAD 10 is placed on the left chest side, another on the right chest side (“slave 1” BSAD 10), one on the left waist side (“slave 2” BSAD 10) and a last one on V6, on the intersection of the 5th left intercostal space and the anterior axillary line (“slave 3” BSAD 10), where the biopotential difference of each point where BSADs 10 have been placed is collected.

Once the master BSAD 10 has been placed on the user, the button 16 incorporated in the device must be kept pressed for a minimum of 3 seconds for the BSAD 10 to be visible to near mobile devices 20 supplied with Bluetooth (see FIGS. 17a, 17b and 17c). The mobile device 20 will first require the user to be signed up, to generate a password, and user's data, such as name, age, sex, weight, height, among others (see FIG. 17a). Then, connection to the BSAD 10 from the mobile device 20 must be established, and the operation mode must be indicated (see FIG. 17b). In the modality in which multiple BSADs 10 are used, “slave” devices are progressively registered, starting with “slave 1” BSAD 10, “slave 2” BSAD 10, “slave 3” BSAD 10, and lastly the “master” BSAD 10. Once the activation procedure has been completed, the mobile device 20 displays a caption indicating that the BSAD or BSADs 10 are “online” and “connected”. It is worth mentioning that, in an alternative modality, connection of two or more BSADs 10 is not necessary, since they can work independently. The quantity and location of the BSADs 10 to be implemented will depend on the user's requirements.

Configuration of the working mode of the BSAD or BSADs 10 will only take place once. Subsequently, when devices are already configured, synchronization among involved BSADs 10 will be automatic for sampling at time intervals. The samples of each BSAD 10, are sent to the mobile device 20, which will subsequently send them to the first server 21.

Once connection has been established, the mobile device 20, by means of an app, sends an indication to the BSAD 10 or “master” BSAD 10 (depending on the modality) to start sampling. In the modality of a single BSAD 10, when the indication is received, it collects a sample of 5,000 data (one piece of data approximately every 2 milliseconds, for a period of approximately 10 seconds), codes it and sends an indication to the mobile device 20, which will be prepared to receive data. On its part, the BSAD 10 starts sending data once the mobile device 20 has sent a signal to the BSAD 10 to start transmitting data. In the modality in which the user employs multiple BSADs 10, the “master” BSAD 10 starts collecting from slave BSADs 10, sending a signal to “slave” BSADs 10 in the same order as they were “registered” in order for them to start sending data to the “master” BSAD 10. Once the number of data (regularly 5000 data) or the data “package” (regularly containing 1250 packages) has been completed, the “master” BSAD 10 indicates to the relevant slave BSAD 10 that is transmitting to interrupt the transmission. Afterwards, a data transmission start indication is sent to the next “slave” BSAD 10. This is repeated until the data packages of each of the “slave” BSADs 10 registered have been collected. Then, since the “master” BSAD 10 contains the data packages, they are transmitted to the mobile device 20, which in turn transmits them to the first server 20. In an alternative modality, “slave” BSADs 10 will transmit the sampled data directly to the mobile device or computer; otherwise, they will be retransmitted by the mesh nodes.

Table 1 presents a small sample of the data collected by the BSAD 10 sent to the mobile device 20 and retransmitted to the first server 21.

TABLE 1

| | |
|---|---|
| 18:48:09 | Connecting to CleverBea8 . . . |
| 18:48:10 | Connected |
| 10:48:21 | CVB8INICIAR_EL_ENVIO |
| 18:48:39 | INIT185A1848183C185D |
| 18:48:39 | 184A1878185C187D |
| 18:48:39 | 187A1888185C185D |
| 18:48:39 | 184A1868186C186D |
| 18:48:39 | 184A1848181C184D |
| 10:48:39 | 182A1848184C187D |
| 18:48:39 | 186A1868186C187D |
| 18:48:39 | 187A1868186C187D |
| 18:48:39 | 187A1898190C192D |
| 18:48:39 | 189A1908191C189D |
| 10:48:39 | 187A1878187C187D |
| 18:48:39 | 187A1868185C185D |
| 10:48:39 | 187A1868188C187D |
| 10:48:39 | 187A1898189C190D |
| 10:48:39 | 189A1908190C190D |
| 10:48:39 | 191A1918191C191D |
| 10:48:39 | 193A1938192C191D |
| 10:48:39 | 191A1988190C189D |
| 10:48:39 | 190A1988189C190D |
| 10:48:39 | 198A1938191C192D |
| 10:48:39 | 193A1898191C190D |
| 10:48:39 | 198A1988190C189D |
| 10:48:39 | 198A1918190C191D |
| 10:48:39 | 191A1988187C188D |
| 10:48:39 | 187A1898188C190D |
| 10:48:40 | 191A1988188C192D |
| 10:48:40 | 191A1988189C191D |
| 10:48:40 | 189A1898189C191D |
| 10:48:40 | 191A1928192C194D |
| 10:48:40 | 191A1918190C189D |
| 10:48:40 | 189A1898191C193D |
| 10:48:40 | 195A1958195C194D |
| 10:48:40 | 194A1928190C189D |

The data package received by the first server 21 are decoded, with which the data of table 1 transform into the data of table 2.

TABLE 2
2.265
2.365
2.33
2.14
1.72
1.08
0.44
−0.2
−0.84
−1.48
−1.745
−1.56
−1.29
−1.075
−0.965
−0.955
−1.035
−1.11
−1.195
−1.25
−1.27
−1.26
−1.25
−1.235
−1.24
−1.24
−1.255
−1.26
−1.275
−1.28
−1.27

Considering that Table 2 contains a sample of the data package (remembering that in a preferred modality packages of 5000 data are used), the package of 5000 data is sent to two different locations in parallel. The first location is to the second server 22, which allows backing up the data package, that can also be consulted on the medical platform. The other location is the third server 23, which is in charge of processing the data packages from the mobile device or computer 20. This server hosts the neural networks in charge of processing the already decoded data packages (such as the data of the sample illustrated in FIG. 2). Said data packages are entered into the first neural network as “Datum” in the input layer, and each piece of data is multiplied by a weight “W” (see FIG. 18) already determined based on “training” repetitions (which will be discussed below) using ECG data deemed “normal” and those representing a heart disease. For each piece of data, a neuron (which for the input layer hosts a “Datum” from table 2) will have an associated weight “W”, which can be seen as the affinity of that neuron in particular with this piece of data. For example, supposing that “Datum 2500” (Piece of data in position 2500 in table 2) is a piece of data indicating arrhythmia, then the weight “W2500” associated with “Datum 2500” will have a very high value. As illustrated in FIG. 18, each neuron of the input layer is multiplied by an already determined weight W value according to the following equation:

$${Y}_{i} = f * ( \sum {W}_{i j} {Datum}_{i} )$$

Where “Yi” is the output of each neuron, f is the activation function, “Wij” is the weight of the connection of datum “i” with neuron “j”, and “Datumi” is the datum i received; activation function “f” is taken from the Sigmoid, which is expressed in the following equation:

$$F ( Datum ) = 1 / ( 1 + {e}^{- D a t u m} )$$

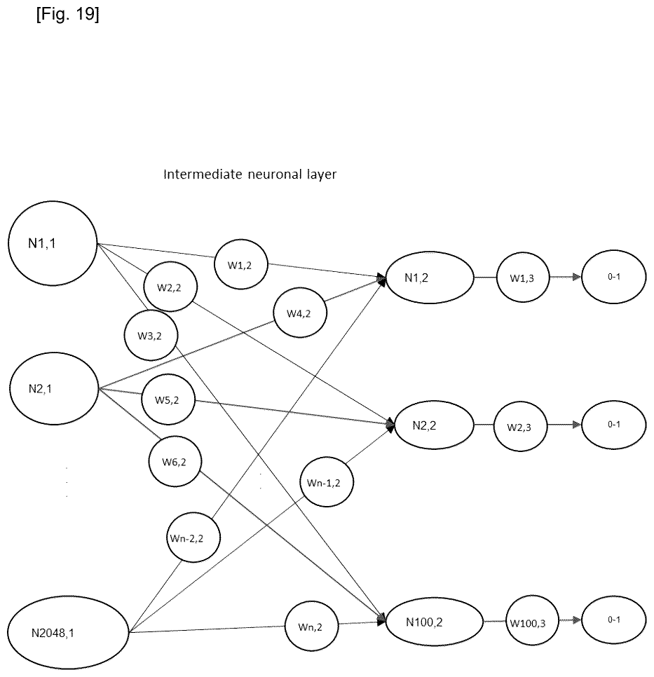

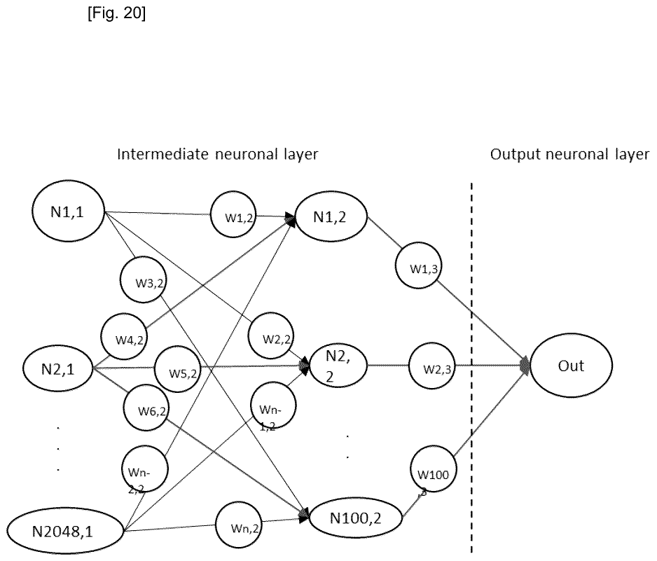

It is worth mentioning that, for activation function “f”, other values different from the Sigmoid expressed above can be used, such as the hyperbolic tangent function (tanh), ReLU, or any other non-linear preferred curve suitable to be used as activation function in neural networks. The output of each “Yi” neuron from the input layer will be a value between 0 and 1, which will tell us how much said neuron was activated. Then, all outputs of the neurons of the input layer will be passed to all the neurons of a first intermediate layer (which comprises the intermediate layers of the neural network). For the purposes of this example, 2048 neurons are used in the first intermediate layer and 100 in the second intermediate layer. Intermediate layers are illustrated in FIGS. 19 and 20. The number of intermediate layers and the number of neurons is determined according to the response precision and speed required by the system. The more layers and neurons, the more the neural network will learn and the better it will be adjusted; however, more processing resources will be required, including a long response time. Therefore, for our neural network exemplified herein, only two intermediate layers and one output layer with one neuron are used (see FIG. 20).

Training of the first and second neural networks can be understood as acquiring a weight “W” database or matrix for each neural network that allows having the correct weights of each parameter, i.e., the correct affinities of each neuron vs. each datum. In order to achieve this, it is necessary to subject each neural network to a training process where data of ECGs whose status is known beforehand are entered (i.e., if it is a healthy signal, its status should be 1 or close to 1; for the second neural network, special attention is paid to the ECGs of the different heart diseases known to medical science, and regarding this particular aspect, each heart disease is cataloged, but, for practical detection purposes, in the case of the first neural network, it must be taken into consideration that a heart disease yields a value of zero or close to zero). Then, decoded training data are entered into the first and second neural networks in order to compare the obtained output vs. the expected output by means of an error function, such as binary cross-entropy, least squares, among others. For example, a training datum of a healthy person is entered into the neural network and it yields a value of 0.3 when the expected output value should be 1; thus, a considerable discrepancy is observed, which is analyzed using the following absolute error function:

$$f ( Datum ) = \vert 0.3 - 1 \vert / 1 = 0.7$$

The magnitude of the error will define how substantial the adjustment of weights “W” will be. This adjustment is performed using a method called stochastic gradient descent, where a random change to the weights “W” is made, evaluating again the neural network in a repetitive manner. If the new result is better, the new weights “W” are saved as a baseline, repeating again until the error function is minimized.

Once the weight “W” databases or matrices have been obtained for each neural network and for each layer thereof, it is possible to process the full data package partially illustrated in table 2 (i.e., the 5000 data) in the first neural network, where the output data of the input layer are passed to the first intermediate layer that, for our example, includes 2048 neurons. Each of these neurons processes the data delivered by the input layer and process them using the summation of input data with a weight “W” matrix according to the equation for “Yi”. This is repeated with the output data of the first intermediate layer, which are useful as input data for the second intermediate layer. In an alternative modality, there can be more intermediate layers, for which the number of neurons of each layer must be calculated and the weight “W” matrix must be obtained, being already trained for each of these subsequent layers. For the purposes of the discussed example, upon output of the first neural network, there is a neuron evaluating the output value of said neuron. Specifically, if such output value of the neuron of the output layer is over 0.7, this means that the processed ECG is “normal”, i.e., that the user's status is “ordinary” and there are no cardiac contingencies or heart diseases to be reported, sending a notification to the second server 22; however, if the value is under 0.7, this means that the user's status is “extraordinary”, so there may be a heart disease. In this case, the decoded data package (partially illustrated in table 2) is subject to the second neural network, with the particularity that this second neural network is comprised of a weight “W” database somewhat diverse compared to the first neural network studied above, in addition to including multiple output neurons; however, for the purposes of this example, it also uses 2048 neurons in its first intermediate layer and 100 neurons in its second intermediate layer. Each neuron of the output layer is related to a heart disease. For the purposes of this example, FIG. 21 illustrates three neurons in the output layer of the second neural network, being clear that the number of neurons of the output layer of the aforementioned second neural network is equal to the number of heart diseases that have been studied and that have defined affinities. Drawing back our attention to the present example and FIG. 21, it is deduced that the output of the NA neuron will indicate the probability that the user has arrhythmia, and its connections with data indicating arrhythmia will have much greater weights “W”. The same is applicable to NT, which indicates the probability of tachycardia, and so on for all supported diseases. This information is processed by statistical means and generates a potential scenario that indicates the most related heart disease or heart diseases, as well as their affinity percentage, also identifying the heart disease with the highest affinity percentage, sending this information to the second server 22. This information is also sent to the first server 21 for storage in the machine learning module for calibration of the classification model. On its part, the server 22 processes the information and issues an alert, which is deployed on the medical platform, notifying the individuals authorized for emergencies, and also being sent to the first server 21, which in turn forwards the alert to the mobile device or computer 20; the latter will display on screen an alert signal, as well as a button that allows knowing that the user has seen the alert (see FIG. 17c). In an alternative modality, it will also be possible to deploy a button indicating if the user is well.

It is important to highlight that, for the User's reference-adjusted model, the more people use the BSAD 10 and do it in an extended manner over time, the more data will be supplied to the database and, simultaneously, the machine learning module for calibration of the classification model of neural networks will be kept under constant “learning”, becoming increasingly more efficient in identifying cardiac anomalies, in addition to customizing its features according to each user individually.

Having described this invention in sufficient detail to enable a technician knowledgeable on the matter to reproduce it, it is found to have a high degree of industrial application and inventive activity. It is worth noting that a technician knowledgeable on the matter will be able to discern alternative modalities of this invention, which must be considered within the scope and spirit of the following CLAIMS.

1. A biopotential signal collection and processing system for monitoring comprising:
at least one biopotential signal acquisition device comprising: a wireless module wherein said wireless module includes a microcontroller with an antenna, said microcontroller and said antenna are powered by a first voltage regulator that adapts the voltage of a battery, said wireless module receives a first signal from an Analog Front End digital block, said Analog Front End digital block is provided with an analog-to-digital converter, and with at least one biopotential sensor, said biopotential sensor acquires biopotential electrocardiogram signals through at least one electrode, said analog-to-digital converter adjusts the biopotential electrocardiogram signals be processed, coded, packaged and wirelessly transmitted by the wireless module to a mobile device or computer;
a first server, said first server receives a coded and packaged signal from the mobile device or computer, said first server decodes and rebuilds the coded and packaged signal, said first server labels the coded and packaged signal for identification, traceability, for subsequent storage, and to simultaneously send a labeled signal to a second server and to a third server;
wherein the second server supports a dedicated medical platform where said labeled signal is stored;
wherein the third server processes the labeled signal using a first neural network and a second neural network that operate in tandem, wherein the first neural network classifies a user's status as ordinary status or extraordinary status wherein the second neural network identifies a profile and an affinity percentage of a heart disease from the extraordinary status.

2. The biopotential signal collection and processing system for monitoring according to claim 1, wherein the first neural network and the second neural network have a data to be processed module, a reference data module and a machine learning module for calibration of a classification model.

3. The biopotential signal collection and processing system for monitoring according to claim 1, wherein the first neural network comprises an output layer with a single neuron, said output layer is calibrated and trained to classify the user's status as the ordinary status or the extraordinary status.

4. The biopotential signal collection and processing system for monitoring according to claim 3, wherein the third server sends a signal to the second server and to the first server when the user's status is classified as ordinary status, wherein the first server and the second server report the user's status on the medical platform and on the mobile device or computer.

5. The biopotential signal collection and processing system for monitoring according to claim 3, wherein the third server processes the biopotential signal in the second neural network when the output layer classifies the user's status as extraordinary status.

6. The biopotential signal collection and processing system for monitoring according to claim 1, wherein the second neural network comprises an output layer with multiple neurons, wherein each of the multiple neurons is related to one heart diseases.

7. The biopotential signal collection and processing system for monitoring according to claim 6, wherein the multiple neurons of the output layer of the second neural network is configured to indicate a probability of the user having a given heart disease, the affinity of each heart disease in the multiple neurons is analyzed, statistically to generate a potential scenario, and to indicate a most related heart disease or heart diseases and the affinity percentage of each heart disease, wherein the heart disease with the highest affinity percentage is identified to determine if it has a high affinity percentage, and to alert about a type of heart disease found.

8. The biopotential signal collection and processing system for monitoring according to claim 7, wherein the third server sends the type of heart disease to the second server and to the first server to storage the type of heart disease in the machine learning module for calibration of the classification model of the first neural network and the second neural network; the second server processes the type of heart disease and issues an alert, said alert is deployed on the medical platform, said alert is configured to notify individuals authorized for emergencies, said second server sends the alert to the first server, said first server forwards the alert to the mobile device or computer to alert the user.

9. The biopotential signal collection and processing system for monitoring according to claim 1, wherein the battery is electrically coupled with a first voltage regulator and with a second voltage regulator, wherein the first voltage regulator is electrically connected to the wireless module, to a button and to the Analog Front End digital block wherein said second voltage regulator is electrically connected to a first bypass capacitor module, to the Analog Front End digital module and to an oscillator electrically connected to the Analog Front End digital module.

10. The biopotential signal collection and processing system for monitoring according to claim 9, wherein the second voltage regulator is controlled by a digital signal issued by the wireless module.

11. The biopotential signal collection and processing system for monitoring according to claim 1, wherein the wireless module of the biopotential signal acquisition device is electrically coupled with the second voltage regulator or the battery.

12. The biopotential signal collection and processing system for monitoring according to claim 1, wherein the biopotential signal acquisition device includes multiple Analog Front Ends.

13. The biopotential signal collection and processing system for monitoring according to claim 9, wherein the wireless module includes an internal, analog-to-digital converter, said internal analog-to-digital converter allows to directly receive at least one biopotential analog signal.

14. A method for acquisition of biopotential data comprising:
starting a wireless module of a first device to activate a Bluetooth™ module and an antenna of the wireless module, allowing to establish a wireless connection to a second device;
sending an indication to the wireless module for configuring peripherals by means of the second device;
enabling a second voltage regulator of the first device to configure sampling parameters of an Analog Front End digital module;
acquiring biopotential signals from the Analog Front End digital module detected by biopotential sensors;
collecting data, the date includes a predetermined quantity of biopotential signals from the Analog Front End digital module via the wireless module;
resetting the wireless module when the collection of the data is completed;
disabling the second voltage regulator of the first device by putting the second voltage regulator into low consumption mode;
sending a signal to the second device through the wireless module to start sending data;
sending a second signal to the wireless module via the second device indicating that the second device is ready to receive data;
starting a timer for sending data;
interrupting the timer via the wireless module when the wireless module is coding and packaging data,
restoring the timer when the wireless module completes coding and packaging of the data;
transmitting coded and packaged data to the second device via the wireless module; and
stopping transmission of the coded and packaged data when the second device reports complete reception of the coded and packaged data.

15. (canceled)

16. A method for processing coded and packaged biopotential data for detection of heart diseases, comprising:
sending coded and packaged data from a mobile device to a first computing server;
decoding and rebuilding the coded and packaged data through the first computing server;
labeling the coded and packaged data for traceability and storing;
sending the labeled data to a second computing server and to a third computing server;
storing the labeled data in the second server;
processing the labeled data through two tandem neural networks from the third computing server in search of heart diseases;
analyzing cardiac rate information stored in the label data to determine cardiac activity status via the third computer server;
adding a second label to the labeled signal according to the cardiac activity status;
storing and sending the cardiac activity status data to the second computing server;
alerting via the second server the existence of a heart disease when the heart disease is detected in the cardiac activity status data by the second computing server;
sending a heart disease alert to the mobile device via the first computing server;
sending the cardiac activity status data from the second server to the third computing server for comparison when the second computing server detects null affinity with a known heart disease;
sending the cardiac activity status data from the second server to the third computing server when a high affinity with a heart disease is detected by the second computer server to compare the cardiac activity status data with a database stored in the third computer server
sending a second alert signal from the third computer server to the second computing server when a high affinity between the heart disease stored in the database and the cardiac activity status data is found, the second computer server issuing a third alert, the second computer server sending the third alert to the mobile device cia the first computing server;
adding a third label to the cardiac activity status data when null affinity with a heart disease was found, indicating that the two comparisons of heart diseases have been carried out;
analyzing cardiac activity status data via a first trained neural network of the third computing server to predict potential heart diseases;
analyzing the cardiac activity status data via a second trained neural network to predict different potential heart diseases associated with a probability of occurrence and a percentage of affinity with one of the heart diseases;
sending a fourth alert to the second computing server when values of the probability have a predetermined value, the second computing server sending the fourth alert to the mobile device via the first computing server.

17.-18. (canceled)

19. The biopotential data processing method is according to claim 16,
wherein a low probability of heart disease is categorized as a user's ordinary status, and the low probability of heart disease is correlated with a value between 0 and 0.68 of an output neuron of an output layer of the first neural network,
and further wherein a high probability of heart disease is associated with a user's extraordinary status, and the high probability of heart disease is correlated with a value between 0.7 and 1 of the output neuron.

20. (canceled)

21. The biopotential data processing method is according to claim 16, wherein the first trained neural network has a single intermediate neuron layer, wherein a number of neurons of the single intermediate neuron layer is between 10% and 50% the number of neurons of an input layer.

22. The biopotential data processing method is according to claim 16, wherein the first trained neural network has two intermediate neuron layers, wherein the number of neurons of the first intermediate neuron layer is between 30% and 50% the number of neurons of an input layer, the second intermediate neuron layer has between 3% and 20% the number of neurons of the first intermediate neuron layer, with which the number of neurons per layer is decreased.

23. (canceled)

24. (canceled)

25. The method for acquisition of biopotential data set forth in claim 14, wherein the first device is a biopotential signal acquisition device, wherein the second device is a mobile device.

26. The method for acquisition of biopotential dataset forth in claim 14, wherein the first device is a first biopotential signal acquisition device, wherein the second device is a second biopotential signal acquisition device.
